(19) United States
(12) Patent Application Publication
(10) Pub. No.: US 2013/0345177 A1
(43) Pub. Date: Dec. 26, 2013

(54) Oleyl Phosphocholine for the Treatment of Mycosis

(71) Applicant: Bruno Jansen, Beerse (BE)

(72) Inventors: Frans Herwig Jansen, Oud-Turnhout (BE); Annie Marie Forten, Montreal (CA)

(73) Assignee: DAFRA PHARMA RESEARCH & DEVELOPMENT BVBA, Turnhout (BE)

(21) Appl. No.: 14/003,046

(22) PCT Filed: Feb. 24, 2012
(86) PCT No.: PCT/EP2012/053144

§ 371 Date: Sep. 4, 2013

(30) Foreign Application Priority Data
Mar. 4, 2011 (EP) PCT/EP2011/053345

(51) Int. Cl. A61K 31/685

(52) U.S. Cl. CPC A61K 31/685
USPC 514/114

(57) ABSTRACT

The present invention relates to the use of oleyl phosphocholine (C18:1-PC), or OlPC,for the treatment of mycosis, and especially for the treatment of mycosis such as mycosis caused by pathogens belonging to a genus selected from the group consisting of Candida, Aspergillus, Fusarium, Cryptococcus, Microsporum, Sporothrix, Trichophyton and Scedosporium, for example, Candida albicans, Candida parapsilosis, Candida glabrata, Candida krusei, Aspergillus fumigatus, Aspergillus niger, Aspergillus terreus, Fusarium solani, Scedosporium prolifacans, Cryptococcus neoformans, Microsporum canis, Sporothrix schenkii, Trichophyton rubrum, Trichophyton mentagrophytes, Aspergillus fumigatus, Fusarium oxysporum.

The present invention relates to the treatment of mycosis, and especially the treatment of virulent or invasive mycosis such as candidemia, cryptococcosis or aspergillosis.

Mycosis is a fungal infection of vertebrates, including humans. Mycosis is a common infection, and a variety of environmental and physiological conditions can contribute to the development of mycosis. Inhalation of fungal spores or localized colonization of the skin may initiate persistent infections and, accordingly, mycosis often starts in the lungs or on the skin.

Mycosis can be classified according to the tissue initially colonized.

Superficial mycosis is limited to the outermost layers of the skin and hair. An example of superficial mycosis is Tinea versicolor, a fungus infection that commonly affects young people.

Cutaneous mycosis extends deeper into the epidermis and also includes invasive hair and nail diseases. These diseases are restricted to the keratinized layers of the skin, hair, and nails. Unlike the superficial mycosis, host immune responses may be evoked resulting in pathologic changes expressed in the deeper layers of the skin.

Subcutaneous mycosis involves the dermis, subcutaneous tissues, muscle, and fascia. These infections are chronic and can be initiated by piercing trauma functioning as a port of entry for the fungi. These infections are difficult to treat and may require surgical interventions such as debridement.

Systemic mycosis generally starts with infection of the lungs, but also other ports of entry are known, and may spread to many organ systems and tissues. Pathogenic fungi causing systemic mycosis are inherently virulent.

A special class of systemic mycosis is systemic mycosis caused by opportunistic pathogens. Systemic mycosis caused by opportunistic pathogens is often an infection in patients with immune deficiencies. Examples of immunocompromised conditions include AIDS, alteration of normal flora by antibiotics, immunosuppressive therapy and metastatic cancer. Examples of opportunistic mycosis include candidiasis, cryptococcosis and aspergillosis.

Pathogenic fungi and especially pathogenic fungi causing systemic mycosis are, for example, fungi belonging to the genus Candida, the genus Aspergillus and the genus Cryptococcus.

Candida species, such as Candida albicans, are major human pathogens that are known for causing opportunist infections in immunocompromised hosts such as transplant patients, patients suffering from AIDS and cancer patients. Infections are difficult to treat and can be very serious, i.e. 30% to 40% of the systemic infections result in death.

Aerosolized Aspergillus spores are found nearly everywhere and constant exposure to Aspergillus is common. Aspergillus can generally cause serious mycosis in three ways: through the production of mycotoxins; through induction of allergenic responses; and through systemic infections.

The most common pathogenic species are Aspergillus fumigatus and Aspergillus flavus. Aspergillus flavus produces an aflatoxin which is both a toxin and a carcinogen.

Mycosis caused by Aspergillus is generally designated as aspergillosis. The symptoms of aspergillosis include fever, cough, chest pain or breathlessness.

Cryptococcus neoformans can cause a severe form of meningitis and meningo-encephalitis for example in HIV positive patients and AIDS. Cryptococcus neoformans is a major human and animal pathogen. Cryptococcus laurentii and Cryptococcus albidus have been known to cause moderate-to-severe disease in human patients with compromised immunity. Cryptococcus gattii can cause disease in non-immunocompromised people.

Presently, amphotericin B, voriconazole and itraconazole are amongst the most commonly used medicaments to combat severe forms of mycosis such as subcutaneous mycosis and systemic mycosis. However, serious side effects are associated with these drugs.

Amphotericin B is a polyene anti-fungal drug often used intravenously for systemic mycosis. It was originally extracted from Streptomyces nodosus, a filamentous bacterium, in 1955.

Two amphotericins, amphotericin A and amphotericin B are known, but only B is used clinically, because it is significantly more active in vivo. Amphotericin A is almost identical to amphotericin B (having a double C═C bond between the 27th and 28th carbon), but has little anti-fungal activity has been observed.

Currently, the drug is available as plain amphotericin B, a cholesteryl sulfate complex, a lipid complex, and as a liposomal formulation.

One of the main intravenous uses of amphotericin B is in treating various systemic mycoses, including cryptococcal meningitis, for example in critically ill, comorbidly infected or immunocompromised patients.

Another intravenous use of amphotericin B is as a drug of last resort in otherwise untreatable parasitic protozoan infections such as visceral leishmaniasis and primary amoebic meningoencephalitis.

Amphotericin B is well-known for its severe and potentially lethal side effects. Very often, a serious acute reaction after the infusion (1 to 3 hours later) is noted, consisting of high fever, shaking chills, hypotension, anorexia, nausea, vomiting, headache, dyspnea and tachypnea, drowsiness, and generalised weakness. To decrease the likelihood and severity of the symptoms, initial doses should be low, and increased slowly.

Intravenously administered amphotericin B has also been associated with multiple organ damage in therapeutic doses. Nephrotoxicity (kidney damage) is a frequently reported side effect and can be severe and/or irreversible.

Voriconazole (VFEND) is a triazole anti-fungal medication that is generally used to treat virulent or invasive mycosis. This type of mycosis is generally seen in patients who are immunocompromised, and includes invasive candidiasis, invasive aspergillosis, and certain emerging fungal infections.

Voriconazole has become the new standard of care in the treatment of invasive aspergillosis, which may occur in immunocompromised patients, including allogeneic BMT, other hematologic cancers, and solid organ transplants.

Voriconazole is better tolerated than amphotericin B, with fewer adverse effects and a longer duration of therapy.

Voriconazole has proven to be as effective as a regimen of intravenous amphotericin B followed by oral fluconazole in patients with culture-proven candidemia. Voriconazole cleared Candida yeast from the bloodstream as quickly as amphotericin B (median 2 days) and showed a trend toward better survival. Voriconazole is also associated with fewer serious adverse events and cases of renal toxicity, but a higher incidence of visual disturbances was observed.

Voriconazole has also been proven effective against a number of other serious fungal pathogens. These include infections by Fusarium spp. and Scedosporium apiospermum (asexual form of Pseudallescheria boydii).

Although infrequently seen in the present clinical setting, these fungi are emerging as more common and deadly causes of fungal infection in immunocompromised patients, and the development of voriconazole has been an important advance in their treatment as they are generally resistant to other antifungal agents (including amphotericin B).

The most common side effects associated with voriconazole include transient visual disturbances, fever, rash, vomiting, nausea, diarrhea, headache, sepsis, peripheral edema, abdominal pain, and respiratory disorder. Also cases of serious hepatic reactions during treatment with voriconazole have been reported.

Itraconazole is a triazole anti-fungal agent that is prescribed to patients with mycosis. The drug may be given orally or intravenously.

Itraconazole has a broader spectrum of activity than fluconazole (but not as broad as voriconazole or posaconazole). In particular, it is active against Aspergillus, which fluconazole is not. It has been approved for the treatment of blastomycosis, histoplasmosis and onychomycosis. It is also prescribed for systemic infections such as aspergillosis, candidiasis and cryptococcosis where other antifungal drugs are inappropriate or ineffective.

Itraconazole is a relatively well-tolerated drug (although not as well tolerated as fluconazole or voriconazole) and the range of adverse effects it produces is similar to the other azole anti-fungals.

Elevated alanine aminotransferase levels are found in 4% of people taking itraconazole forming a risk of developing congestive heart failure. Other reported adverse effects of itraconazole are nausea, vomiting, abdominal pain, fatigue, loss of appetite, yellow skin (jaundice), yellow eyes, itching, dark urine, and/or pale stool.

Considering the above, there is a need in the art for anti-fungal agents being similarly effective as amphotericin B, voriconazole and/or itraconazole with respect to anti-fungal activity but which are better tolerated, i.e. with less, reduced or less severe side effects.

Further, considering the rapid development of resistance against known anti-fungal compounds, there is a continuing need in the art for further compounds with anti-fungal activity, and especially anti-fungal compounds effective against life threatening invasive or virulent fungal pathogens.

Accordingly, it is an object of the present invention, amongst other objects, to provide anti-fungal compounds meeting the above needs in the art.

According to the invention, this object, amongst other objects, according to a first aspect is met by appended claim 1.

Specifically, this object, amongst other objects, according to a first aspect is met by oleyl phosphocholine for use in the treatment of mycosis in a vertebrate, preferably for use in the treatment of invasive or virulent mycosis in a vertebrate.

Virulent mycosis as used herein indicates a primary fungal infection capable of extending, or spreading, the infected area of an organ or tissue infected.

Invasive mycosis as used herein indicates a primary fungal infection capable of further infecting, or spreading to, other organs or tissues.

In the present context, mycosis can be both virulent and invasive.

According to a preferred embodiment of this aspect of the present invention, the present mycosis is selected from the group consisting of superficial mycosis, cutaneous mycosis, subcutaneous mycosis, and systemic mycosis and especially invasive or virulent subcutaneous mycosis or invasive or virulent systemic mycosis.

According to another preferred embodiment of this aspect of the present invention, the present vertebrate is a human, preferably an immunocompromised patient.

According to yet another preferred embodiment of this aspect of the present invention, the causative pathogen of the present mycosis is selected from the genus Candida, Aspergillus, Fusarium, Cryptococcus, Microsporum, Sporothrix, Trichophyton and Scedosporium.

According to an especially preferred embodiment of this aspect of the present invention, the present oleyl phosphocholine is used for the treatment of mycosis caused pathogens selected from the group consisting of Candida albicans, Candida parapsilosis, Candida glabrata, Candida krusei, Aspergillus fumigatus, Aspergillus niger, Aspergillus terreus, Fusarium solani, Scedosporium prolifacans, Cryptococcus neoformans, Microsporum canis, Sporothrix schenkii, Trichophyton rubrum, Trichophyton mentagrophytes, Aspergillus fumigatus, Fusarium oxysporum.

According to another preferred embodiment of this aspect of the present invention, the present oleyl phosphocholine is formulated for parenteral, oral or topical administration. Especially in case the present mycosis is subcutaneous mycosis oleyl phosphocholine is formulated for topical or oral administration and in case the present mycosis is systemic mycosis oleyl phosphocholine is formulated intravenous, parenteral or oral administration.

According to an especially preferred embodiment of the present invention, the present oleyl phosphocholine is used for the treatment of a mycosis selected from the group consisting of candidemia, aspergillosis and cryptococcosis.

Considering the effective anti-fungal activity of oleyl phosphocholine, the present invention, according to another aspect, relates to methods for treatment of mycosis comprising administering to, or applying on a vertebrate suffering from mycosis, preferably invasive or virulent mycosis, a therapeutically effective dose of oleyl phosphocholine.

According to a preferred embodiment of this aspect, the present mycosis is caused by a yeast or fungus belonging to a genus selected from the group consisting of Candida, Aspergillus, Fusarium, Cryptococcus, Microsporum, Sporothrix, Trichophyton and Scedosporium such as pathogens selected from the group consisting of Candida albicans, Candida parapsilosis, Candida glabrata, Candida krusei, Aspergillus fumigatus, Aspergillus niger, Aspergillus terreus, Fusarium solani, Scedosporium prolifacans, Cryptococcus neoformans, Microsporum canis, Sporothrix schenkii, Trichophyton rubrum, Trichophyton mentagrophytes, Aspergillus fumigatus, Fusarium oxysporum.

According to yet another preferred embodiment of this aspect, the present invention relates to the present use of oleyl phosphocholine formulated by parenteral, oral or topical administration.

According to a particularly preferred embodiment of this aspect, oleyl phosphocholine is used for the treatment of candidemia, aspergillosis and cryptococcosis.

The present invention will be further detailed and illustrated in the examples below representing particularly preferred embodiments of the present invention.

### EXAMPLES

### Example 1

The following ten strains were tested for assaying the anti-fungal activity of oleyl phosphocholine. One Candida strain, i.e. 19114, was selected because of its resistance against voriconazole and itraconazole

TABLE 1
Strain tested for anti-fungal activity
of oleyl phosphocholine

| Strain | strain isolate | remark |
|---|---|---|
| Yeasts | | |
| Candida albicans | 3731 | ATCC 10231 |
| Candida albicans | 19114 | clinical blood |
| | | isolate resistant |
| | | for voriconazole |
| | | and itraconazole |
| Candida parapsilosis | 3270 | ATCC 22019 |
| Candida glabrata | 19371 | clinical blood |
| | | isolate |
| Candida krusei | 9560 | ATCC 6258 |
| filamentous fungi | | |
| Aspergillus fumigatus | 18963 | NCPF 7367 |
| Aspergillus niger | 5788 | ATCC 10864 |
| Aspergillus terreus | 13669 | clinical isolate |
| Fusarium solani | 18489 | clinical isolate |
| Scedosporium | 22387 | clinical blood |
| prolifacans | | isolate |

The above strains were contacted with oleyl phosphocholine on microplates and the anti-fungal activity of oleyl phosphocholine was determined. In comparison, the same test was performed using amphotericin B (AMB), voriconazole (VRZ) and itraconazole (ITR).

The test protocol used was in conformity with the standard CLSI method:

Clinical and Laboratory Standards Institute/National Committee for Clinical Laboratory Standards (2002) Reference method for broth dilution antifungal susceptibility testing of yeasts. Approved Standard document M27-A2. Clinical and Laboratory Standards Institute/National Committee for Clinical Laboratory Standards Institute, Wayne Pa.
Clinical and Laboratory Standards Institute/National Committee for Clinical Laboratory Standards. (2002) Reference method for broth dilution antifungal susceptibility testing of filamentous fungi. Approved Standard document M38-A. Clinical and Laboratory Standards Institute/National Committee for Clinical Laboratory Standards Institute, Wayne

The following concentrations of oleyl phosphocholine (Dafra), amphotericin B (AMB), voriconazole (VRZ) and itraconazole (ITR) were tested: 10, 5, 2.5, 1.25, 0.625, 0.312, 0.15, 0.078, 0.039, 0.019, 0.009, and 0.004 μg/ml

The results, after visual and spectrophometric reading after 48 hours of incubation, are presented as MIC50 and MIC 100, i.e. the minimum inhibitory concentration required inhibiting the growth of 50% or 100% of the microorganisms, respectively.

TABLE 2
Minimum inhibitory concentration required inhibiting the
growth of 50% or 100% of the microorganisms

| | | Dafra- | | | |
|---|---|---|---|---|---|
| | | MIC100 | ITR | VRZ | AMB |
| | IHEM | (48 h) | (MIC50) | (MIC50) | (MIC100) |
| Yeast | N^{o} | (μg/ml) | (μg/ml) | (μg/ml) | (μg/ml) |
| Candida albicans | 3731 | 2.5 | 0.032 | 0.064 | 0.5 |
| Candida albicans | 19114 | 2.5 | >16 | >16 | 0.5 |
| Candida | 3270 | 5 | 0.064 | 0.125 | 1 |
| parapsilosis | | | | | |
| Candida glabrata | 19371 | 5 | 0.5 | 0.25 | 0.5 |
| Candida krusei | 9560 | 5 | 0.25 | 0.5 | 1 |
| | | Dafra- | | | |
| | | MIC100 | ITR | VRZ | AMB |
| | IHEM | (48 h) | (MIC100) | (MIC100) | (MIC100) |
| Fungi | N^{o} | (μg/ml) | (μg/ml) | (μg/ml) | (μg/ml) |
| Aspergillus | 18963 | 5 | 0.5 | 1 | 2 |
| fumigatus | | | | | |
| Aspergillus niger | 5788 | 10 | 0.5 | 1 | 1 |
| Aspergillus | 13669 | 10 | 0.25 | 0.5 | >8 |
| terreus | | | | | |
| Fusarium solani | 18489 | 5 | >16 | 16 | 2 |
| Scedosporium | 22387 | 5 | >16 | 16 | >8 |
| prolificans | | | | | |

As can be seen in the above table, oleyl phosphocholine was capable of inhibiting the growth of yeast and fungi in a similar concentration range as the commonly used anti-fungal agents amphotericin B, voriconazole and itraconazole.

Further, the results for the resistant Candida strain 19114 show that oleyl phosphocholine provides a suitable alternative for the known anti-fungal agents against which resistance has been developed.

Of special interest is the anti-fungal activity of oleyl phosphocholine against Fusarium solani and Scedosporium prolificans, two emerging pathogens, against at least two of the known anti-fungal agents, i.e. voriconazole and itraconazole, are less effective.

### Example 2

The following strains were tested for assaying the anti-fungal activity of oleyl phosphocholine.

TABLE 3
Strains used for assaying the anti-fungal
activity of oleyl phosphocholine.

Candida albicans
Candida albicans B59630 (R)
Candida albicans B63195 (S)
Candida parapsilosis B66126
Cryptococcus neoformans
Cryptococcus neoformans B66663
Microsporum canis
Sporothrix schenkii
Sporothrix schenkii B62482
Trichophyton rubrum
Trichophyton rubrum B68183
Trichophyton mentagrophytes B70554
Aspergillus fumigatus
Aspergillus fumigatus B42928
Aspergillus fumigatus B19119
Fusarium solani IHEM22128
Fusarium oxysporum IHEM3014

The above strains were contacted oleyl phosphocholine on microplates and the anti-fungal activity of oleyl phosphocholine was determined.

Anti-fungal activity was expressed as IC₅₀, i.e. the concentration oleyl phosphocholine needed to kill 50% of the pathogens. The results are summarised in Table 4 below:

TABLE 4
In vitro efficacy of OlPC against yeasts and fungi
expressed as IC₅₀ concentration.

| | OlPC |
|---|---|
| | IC₅₀ |
| Yeasts/Fungi | (μM) |
| Candida albicans | 7.89 |
| Candida albicans B59630 (R) | 8.00 |
| Candida albicans B63195 (S) | 7.77 |
| Candida parapsilosis B66126 | 8.00 |
| Cryptococcus neoformans | 4.50 |
| Cryptococcus neoformans B66663 | 0.50 |
| Microsporum canis | 1.66 |
| Sporothrix schenkii | 8.16 |
| Sporothrix schenkii B62482 | 1.97 |
| Trichophyton rubrum | 5.87 |
| Trichophyton rubrum B68183 | 2.00 |
| Trichophyton mentagrophytes B70554 | 2.00 |
| Aspergillus fumigatus | 18.76 |
| Aspergillus fumigatus B42928 | 8.00 |
| Aspergillus fumigatus B19119 | 1.87 |
| Fusarium solani IHEM22128 | 2.31 |
| Fusarium oxysporum IHEM3014 | 2.55 |

1-10. (canceled)

11. A method for treatment of mycosis comprising administering to a vertebrate suffering from mycosis, a therapeutically effective dose of oleyl phosphocholine

12. The method according to claim 11, wherein said mycosis is selected from the group consisting of superficial mycosis, cutaneous mycosis, subcutaneous mycosis, and systemic mycosis.

13. The method according to claim 11, wherein said mycosis is at least one of invasive subcutaneous mycosis, virulent subcutaneous mycosis, invasive systemic mycosis, or virulent systemic mycosis.

14. The method according to claim 11, wherein said vertebrate is a human.

15. The method according to claim 11, wherein said mycosis is caused by a pathogen selected from the group consisting of Candida, Aspergillus, Fusarium, Cryptococcus, Microsporum, Sporothrix, Trichophyton and Scedosporium.

16. The method according to claim 11, wherein said mycosis is caused by a pathogen selected from the group consisting of Candida albicans, Candida parapsilosis, Candida glabrata, Candida krusei, Aspergillus fumigatus, Aspergillus niger, Aspergillus terreus, Fusarium solani, Scedosporium prolifacans, Cryptococcus neoformans, Microsporum canis, Sporothrix schenkii, Trichophyton rubrum, Trichophyton mentagrophytes, Aspergillus fumigatus, and Fusarium oxysporum.

17. The method according to claim 11, wherein said oleyl phosphocholine is administered parenterally, orally, or topically.

18. The method according to claim 11, wherein said mycosis is systemic mycosis and said oleyl phosphocholine is administered intravenously or orally.

19. The method according to claim 11, wherein said mycosis is subcutaneous mycosis and said oleyl phosphocholine is administered topically or orally.

20. The method according to claim 11, wherein said mycosis is selected from the group consisting of candidemia, aspergillosis and cryptococcosis.

21. The method according to claim 14, wherein the human is immunocompromised.
